SEQUENCE LISTING (2 sequences)

SEQ ID NO: 1 - DNA, 1230 bp, Homo sapiens
attcagtagactttggtcctgttcacaaaagctaggggtggctagatggctagacaaaccatggaatgggaagggaagtgtgttgcagttgcaggcagaagcatgaaggggatgggacaaaagaggcggtggcaagatcttagatgcccacgagtgccaagaaagcaggtgggcagacctgcctgtagggaggcctcgacgcttgacacgcccgacactgtgccctgtgtcctcggcacgtggcgagggcggccagggccaggcgcagtgacgggcgcggcagccgggccggggtgcggggcacgggctgccctcatgccctcgcgtcttcccccaggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtgagggcacagcgggccccagggctaccctcccccaggaggtcgaccccaaagccccttgctctcccctgccctgctgccgcctcccagcctggggggtcgtggcagataatcagcctcttaaagctgcctgtagttaggaaataaaacctttcaaatttcacatccacctctgactttgaatgtaaaccgtgtgaataaagtaaaaaatacgtagccgcaata

SEQ ID NO: 2 - DNA, 91 bp, Homo sapiens
tacggcggtttcatgacctccgagaagagccagacgcccctggtgacgctgttcaaaaacgccatcatcaagaacgcctacaagaagggcg

(19) United States
(12) Patent Application Publication
(10) Pub. No.: US 2002/0064833 A1
(43) Pub. Date: May 30, 2002

(54) Recombinant plasmids and method for treating substance abuse

(76) Inventors: Tore H. Fagerlund, Osteras (NO); Peter Alestrom, Oslo (NO); Kare Berg, Oslo (NO)

Correspondence Address: CHRISTIAN D. ABEL, POSTBOKS 333 SENTRUM, BERGEN, N-5804, NO

(21) Appl. No.: 09/960,288

(22) Filed: Sep. 24, 2001

Related U.S. Application Data
(63) Continuation of application No. 09/426,877, filed on Oct. 26, 1999, now abandoned.

(30) Foreign Application Priority Data
Oct. 26, 1998 (NO) 19984992

(51) Int. Cl. C07K 14/675; C12Q 1/68; C07H 21/04; C12N 5/06

(52) U.S. Cl. 435/69.4; 530/399; 435/320.1; 435/325; 536/23.5

(57) ABSTRACT

The present invention relates to a polynucleotide for use in therapy directed at the peripheral and central nervous system in humans for the purpose of treating psychological dependence on, and abuse of, substances which have a stimulating and euphoric effect. In particular, the invention relates to a recombinant plasmid comprising a plurality of sequences that encode for beta-endorphin, said sequences being separated by a proteolytic cleavage site.

### CROSS-REFERENCE TO RELATED APPLICATION

This is a continuation of application Ser. No. 09/426,877, filed Oct. 26, 1999.

### TECHNICAL FIELD

The present invention relates to a polynucleotide for use in therapy directed at the peripheral and central nervous system in humans for the purpose of treating psychological dependence on, and abuse of, substances which have a stimulating and euphoric effect. In particular, the invention relates to a recombinant plasmid encoding for beta-endorphin and treatment method.

### BACKGROUND

Dependence on, and abuse of, addictive substances is currently treated with medications administered orally or parenterally. The medications used in this connection consist of foreign matter with special pharmacological characteristics and effects for each individual who takes them. Some of the characteristics and effects of these medications are desirable, while others are less desirable (side effects).

The genetic-engineering approach to treatment of disease conditions involves altering the expression of genes that are already present, in such a way as to increase or reduce the amount of this particular genetic product, or it can involve the introduction of modified (improved) versions of the existing genes so that the genetic product becomes (qualitatively or quantitatively) different (better) than the original genetic product. Research in this area is still in its early days, but it has already reached the stage in the USA where gene therapy for particular human diseases is being tested. These genetic modifications are not hereditary. The method entails the introduction of a gene construct into the spinal fluid, blood or somatic tissue in humans and animals. The gene construct's DNA is taken up by cells in the respective tissue, and remains there in the form of extra-chromosomal DNA expressed as mRNA and protein (genetic expression). No one has yet been able to demonstrate that gene constructs of this type can be incorporated within chromosomes. Even if this were to occur, the characteristics could not be hereditary because they will not be present in the sex cells.

### RESEARCH IN THE AREA OF PSYCHOLOGICAL DEPENDENCE ON SUBSTANCES

Data has been published which forms the basis for regarding psychological dependence on substances as a complex neurophysiological condition involving endogenous opioids (Goldstein 1976, 1983). The authors of this published data indicate that a lack of endogenous opioids or an abnormal function in the endogenous opioid systems may be the cause of the development of addictive behavior. The endogenous opioid systems are subject to genetic factors to a considerable degree. It has been known for a long time that beta-endorphins have powerful analgesic characteristics in both humans and animals (Loh et al 1976, Feldberg & Smith 1977).

More recent research has also shown that the presynaptic release of beta-endorphins from fibers originating in the hypothalamus causes a continuous reward tonus (Mucha, Millian and Herz, 1985). These research findings fit in with data from other experiments which indicate the important role that these endogenous opioid systems play in reward processes (van Ree, Smyth and Colpaert 1983; Dum and Hertz 1987; Sweep et al. 1989). There is increasing evidence to show that endogenous opioids are involved in the effects of stimulants such as cocaine and amphetamine, as well as nicotine and depressants such as alcohol and benzodiazepines (Spyraki and Fibiger 1988; Carboni et al 1989; Koob 1992). Both behavioral studies and biochemical data indicate connections between stimulants and endogenous opioid mechanisms (Mello et al 1989; Spealman and Bergman 1992; Jones and Holtzman 1992). A number of research results demonstrate the connection between alcohol and endogenous opioids (Olson, Olson and Kastin 1992).

The POMC (pro-opio-melano-cortin) gene has 3 exons and codes for a number of peptides, including exon 3 (SEQ ID NO:1) which encodes for beta-endorphin among others (Tsukada et al 1982). It is known that the analgesic effect of beta-endorphins is obtained by the release of met-enkephalin which stimulates delta-opioid receptors in the spinal cord. It is also known that white blood cells produce beta-endorphins in inflamed tissue which have analgesic and anti-inflammatory properties.

### OBJECTS AND BENEFITS OF THE INVENTION

By means of a novel gene construct the invention provides for a method of genetically altering the reward-giving characteristics in the human central nervous system for the purpose of treating dependence on, and abuse of, addictive substances, without these altered characteristics being hereditary and without the treated individuals becoming transgenic (GMO) (Genetically Modified Organisms).

The gene construct used in this method enables the treatment of psychological dependence on, and abuse of, addictive substances in situations where the present conventional therapies do not adequately meet the objectives of the treatment.

The gene construct used in the method does not entail the genetic modification of sex cells, so no individuals will become transgenic. This method is based on the ability to stimulate a significantly increased production of beta-endorphins by introducing the gene construct as described into the organism. This means that intervention is being made in the central nervous system's reward mechanisms, mechanisms which probably play a central role in the development and maintenance of psychological dependence on, and abuse of, addictive substances.

These altered characteristics are not hereditary, and the period during which the foreign genes are present in the treated individual will be limited. People who are treated with the genetic structure that is employed will not suffer any genetic modification of their sex cells. The characteristics which are affected by the gene construct employed are the characteristics of dependence on, and abuse of, absorbed substances. The gene construct employed achieves this by enabling an increased expression of beta-endorphins in the central nervous system, or an increased expression of beta-endorphins in other tissues, depending on the method of application. Beta-endorphins are among the organism's most potent opioid-like substances, and they play a central role in the organism's analgesic systems both peripherally and centrally, while also having a specific reward effect in the central nervous system. By increasing the production of beta-endorphins, the need to take other substances which affect these mechanisms will diminish or disappear. This means in practice that people will find it easier to break addictive habits without having to use methadone for example, while at the same time avoiding withdrawal symptoms. In other words, the individual's reward mechanism will be normalized over a period of time without the use of other medications and without the possibility of these DNA medications being redistributed, and in such a way that the effect will last for a long time before any possible new treatment may be necessary.

### SUMMARY OF THE INVENTION

The present invention provides for a novel polynucleotide and a special variant of gene therapy which is essentially distinct from transgenic technologies, and which involves an alteration of the characteristic of dependence on, and abuse of, absorbed substances which have a stimulating and euphoric effect. The polynucleotide of the invention comprises a recombinant plasmid containing a plurality of copies of that part of the POMC gene, exon 3 (SEQ ID NO: 1) which codes for beta-endorphins (SEQ ID NO: 2). Each beta-endorphin coding sequence (SEQ ID NO: 2) is separated by a sequence which codes for a proteolytic cleavage site. The method of the invention comprises administering the gene construct according to the invention to a patient.

### DETAILED DESCRIPTION OF THE INVENTION

The target cells for genetic treatment are nerve cells and all other cells that can express the gene construct. Administration of gene constructs will be based on the same principles as those used in gene therapy. Gene constructs will be injected into blood, into spinal fluid, directly into nerve cells or introduced into other tissue by appropriate means. The method will employ a gene construct which is bound to compounds which promote the absorption and expression of the gene construct.

The gene construct consists of a synthetically created gene which in itself consists of many repeated copies of that part of the POMC gene which codes for beta-endorphins (SEQ ID NO:2). Each beta-endorphin coding sequence is separated by a sequence which codes for a proteolytic cleavage site.

The human pro-opio-melano-cortin (POMC) gene, exon 3, has the following nucleotide sequence (See also SEQ ID NO:1):

ATTCAGTAGACTTTGGTCCTGTTCACAAAAGCTAGGGGTGGCTAGATGGCTAGACAAACCATGG
AATGGGAAGGGAAGTGTGTTGCAGTTGCAGGCAGAAGCATCAAGGGGATGGGACAAAAGAGGCG
GTGGCAAGATCTTAGATGCCCACGAGTGCCAAGAAAGCAGGTGGGCAGACCTGCCTGTAGGGAG
GCCTCGACGCTTGACACGCCCGACACTGTGCCCTGTGTCCTCGGCACGTGGCGAGGGCGGCCAG
GGCCAGGCGCAGTGACGGGCGCGGCAGCCGGGCCGGGGTGCGGGGCACGGGCTGCCCTCATGCC
CTCGCGTCTTCCCCCAGGAGTGCATCCGGGCCTGCAAGCCCGACCTCTCGGCCGAGACTCCCAT
GTTCCCGGGAAATGGCGACGAGCAGCCTCTGACCGAGAACCCCCGGAAGTACGTCATGGGCCAC
TTCCGCTGGGACCGATTCGGCCGCCGCAACAGCAGCAGCAGCGGCAGCAGCGGCGCAGGGCAGA
AGCGCGAGGACGTCTCAGCGGGCGAAGACTGCGGCCCGCTGCCTGAGGGCGGCCCCGAGCCCCG
CAGCGATGGTGCCAAGCCGGGCCCGCGCGAGGGCAAGCGCTCCTACTCCATGGAGCACTTCCGC
TGGGGCAAGCCGGTGGGCAAGAAGCGGCGCCCAGTGAAGGTGTACCCTAACGGCGCCGAGGACG
AGTCGGCCGAGGCCTTCCCCCTGGAGTTCAAGAGGGAGCTGACTGGCCAGCGACTCCGGGAGGG
AGATGGCCCCGACGGCCCTGCCGATGACGGCGCAGGGGCCCAGGCCGACCTGGAGCACAGCCTG
CTGGTGGCGGCCGAGAAGAAGGACGAGGGCCCCTACAGGATGGAGCACTTCCGCTGGGGCAGCC
CGCCCAAGGACAAGCGCstartTACGGCGGTTTCATGACCTCCGAGAAGAGCCAGACGCCCCTG
GTGACGCTGTTCAAAAACGCCATCATCAAGAACGCCTACAAGAAGGGCGstopAGTGAGGGCAC
AGCGGGCCCCAGGGCTACCCTCCCCCAGGAGGTCGACCCCAAAGCCCCTTGCTCTCCCCTGCCC
TGCTGCCGCCTCCCAGCCTGGGGGGTCGTGGCAGATAATCAGCCTCTTAAAGCTGCCTGTAGTT
AGGAAATAAAACCTTTCAAATTTCACATCCACCTCTGACTTTGAATGTAAACCGTGTGAATAAA
GTAAAAAATACGTAGCCGCAATA

The beta-endorphin coding nucleotide sequence is marked as appearing between start and stop, and has the following sequence(See also SEQ ID NO:2):

TACGGCGGTTTCATGACCTCCGAGAAGAGCCAGACGCCCCTGGTGACGCTGTTCAAAAACGCCA
TCATCAAGAACGCCTACAAGAAGGCCG

This sequence will be the coding sequence for beta-endorphin in the gene construct (the plasmid), in addition to other DNA sequences which have regulatory functions (as promoters, for example) and which will ensure that the beta-endorphin sequence is correctly transcribed and expressed.

### EXAMPLES

The gene construct can be used in the treatment of psychological dependence on, and abuse of, addictive substances. Administration of the gene construct in the spinal fluid will lead to its absorption by neighboring cells, which will result in the transient expression of the introduced gene with a consequent increase in the beta-endorphin level. This will involve an increased analgesic effect. Administration into other tissues will lead in a corresponding way to absorption by the cells in this tissue and result in expression with a subsequent increase in the beta-endorphin level.

The following conditions can be appropriate for treatment with the genetic product as described:

Psychological dependence on, and abuse of, nicotine.

Psychological dependence on, and abuse of, alcohol.

Psychological dependence on, and abuse of, opiates and opioids

Psychological dependence on, and abuse of, all other substances, known and unknown, which play on the central nervous system's reward mechanisms.

We claim:

1. An isolated and purified nucleic acid molecule that comprises a plurality of sequences that code for a polypeptide the biological activity of which is connected with the reduction of psychological dependence on, and abuse of, substances.

2. The nucleic acid molecule of claim 1, wherein said polypeptide is beta-endorphin and said coding sequences are a plurality of SEQ ID NO:2.

3. The nucleic acid molecule of claim 2, wherein said nucleic acid molecule is DNA.

4. The nucleic acid molecule of claim 3, wherein said nucleic acid molecule is a recombinant plasmid.

5. The nucleic acid molecule of claim 4, wherein the sequences of SEQ ID NO:2 are separated by a proteolytic cleavage site.

6. A method of expressing a polypeptide in host cells, comprising the steps of:
a) providing a nucleic acid molecule, said nucleic acid molecule comprising a plurality of sequences of SEQ ID NO:2;
b) transferring said nucleic acid molecule to said host cells;
c) providing conditions sufficient for the expression of the polypeptide; and
d) expressing the polypeptide in said host cells.

7. The method of claim 6, wherein said sequences of SEQ ID NO:2 are separated by proteolytic cleavage sites;

8. The method of claim 7, wherein said host cells are the cells of a living human being.

9. A method of treating a patient for substance abuse, comprising the steps of:
a) providing a nucleic acid molecule, said nucleic acid molecule comprising a plurality of sequences of SEQ ID NO:2;
b) administering said nucleic acid molecule to the patient in therapeutically effective doses.

10. The method of claim 9, wherein said sequences of SEQ ID NO:2 are separated by proteolytic cleavage sites;
